(19) United States
(12) Patent Application Publication
(10) Pub. No.: US 2023/0022766 A1
(43) Pub. Date: Jan. 26, 2023

(54) VERSATILE STRATEGY FOR COVALENT GRAFTING OF BIOMOLECULES TO CRYOGELS

(71) Applicants: NORTHEASTERN UNIVERSITY Center for Research Innovation Northeastern Univ,, Boston, MA (US); Stichting Katholieke Universiteit, Nijmegen (NL)

(72) Inventors: Sidi A. Bencherif, Boston, MA (US); Jorieke Weiden, Nijmegen (NL); Carl Figdor, Nijmegen (NL); Martijn Verdoes, Nijmegen (NL)

(21) Appl. No.: 17/783,548

(22) PCT Filed: Dec. 9, 2020
(86) PCT No.: PCT/US20/64031

§ 371 Date: Jun. 8, 2022

Related U.S. Application Data
(60) Provisional application No. 62/945,563, filed on Dec. 9, 2019.

(51) Int. Cl. A61K 47/69; A61K 47/61; A61K 47/60; C12N 5/0783; A61K 35/17

(52) U.S. Cl. CPC A61K 47/6903; A61K 47/61; A61K 47/60; C12N 5/0636; A61K 35/17; C12N 2533/80; C12N 2533/74

(57) ABSTRACT

Disclosed are biocompatible cryogels comprising one or more biomolecules, such as antibodies, protein complexes, enzymes, dna and polysaccharides. Also disclosed are methods of making the cryogels.

### RELATED APPLICATION

This application claims the benefit of priority to U.S. Provisional Patent Application No. 62/945,563, filed Dec. 9, 2019, which is incorporated herein by reference in its entirety.

### BACKGROUND

Biomaterial-based scaffolds are increasingly being applied as 3D culture systems in vitro and as molecular and cellular delivery vehicles in vivo. To support cellular survival, activation and differentiation, cells need to be provided with biomolecular cues that trigger specific signaling pathways. For instance, to facilitate survival and expansion of primary T lymphocytes, biomolecules that trigger T-cell receptor signaling and provide co-stimulatory cues are required. Therefore, biomolecules such as activating antibodies, protein complexes and polysaccharides need to be integrated into biomaterial-based scaffolds. These can be incorporated in various ways, e.g., through physical entrapment or ionic interaction but these strategies do not result in stable or controlled presentation of biomolecules. Instead, covalent attachment is favored to ensure sustained availability of these signals in a controlled manner.

Many biomaterial systems currently used to provide cells with a defined set of cues (e.g. expanding T cells with activating antibodies) are 2D systems, whereas repeatedly it has been shown that 3D systems resemble the natural cellular microenvironment and can improve cellular survival and behavior. Moreover, in vivo 3D systems can ensure localized and sustained availability of molecular cues and cells.

### SUMMARY OF INVENTION

There is an increasing interest in using macroporous scaffolds as they can support cellular migration, infiltration and dispersion in contrast to many nanoporous 3D biomaterial-based scaffolds. Cryogelation is a technique that allows to create macroporous scaffolds with controllable pore sizes. Another major advantage of the polysaccharide-based (e.g., hyaluronic acid or alginate) cryogels described in this application in particular is their unique mechanical characteristics which allow for minimally invasive delivery of pre-formed constructs through injection, as opposed to many other pre-formed 3D polymer scaffolds that need to be surgically implanted. The use of pre-formed cryogels furthermore circumvents problems associated with injectable hydrogels that gel in situ including lack of control over the location of the gel, loss of cargo and a poorly defined macrostructure.

Biomolecules are mostly incorporated into biomaterial-based scaffolds in a non-covalent manner via adsorption, whereas covalent attachment provides more control, prevents (unwanted) release of the biomolecules and may enhance cellular responses. To create 3D biomaterial-based scaffolds that present signals in a spatiotemporally controlled manner, methods are required that support covalent attachment of biomolecules to scaffolds while preserving their biological activity. For these type of cryogels, methacrylation of biomolecules has been performed to allow covalent integration during cryopolymerization. This process may hamper bioactivity of biomolecules as they are exposed to free radicals but also may get buried within the polymer walls, preventing their presentation externally on the scaffolds.

In certain embodiments, the present invention provides a polymer comprising a moiety of formula (I):

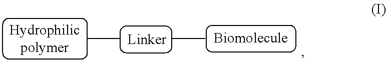

wherein the hydrophilic polymer is crosslinked to one or more additional hydrophilic polymer molecules, and the linker is covalently attached to the hydrophilic polymer.

In certain aspects, the hydrophilic polymer is a polysaccharide. In certain aspects, the polysaccharide is hyaluronic acid or alginic acid.

In certain aspects, the biomolecule is capable of promoting cell expansion.

In further aspects, the present invention provides a cryogel comprising a polymer of the invention.

In certain embodiments, the present invention provides a method of making a cryogel, comprising crosslinking a hydrophilic polymer in an aqueous solvent to generate a crosslinked polymer. In certain embodiments, the hydrophilic polymer is an acrylated or methacrylated polysaccharide. In certain aspects, the acrylated or methacrylated polysaccharide is contacted with a radical initiator in the presence of an acrylate or methacrylate co-monomer.

In further aspects, the present invention provides a formulation comprising a cryogel of the invention and a pharmaceutically acceptable carrier.

The present invention also provides a method of delivering activated T-cells to a tissue, comprising contacting the tissue with a formulation or cryogel of the invention.

### BRIEF DESCRIPTION OF THE DRAWINGS

FIG. 1 relates to pre-formed cryogels are macroporous, injectable and support cell survival. (A) Schematic overview of the cryogelation process to produce injectable cryogels. Polysaccharide polymers (alginate or hyaluronic acid) are chemically modified to create methacrylated polysaccharide polymers that are sensitive to free radical polymerization (1); Methacrylated polymers are dissolved in water (2); Free radical polymerization is triggered before freezing at −20° C. to induce ice crystal formation. The ice crystals exclude the methacrylated polymers (3); after the crosslinking of methacrylated polymers concentrated around ice crystals, thawing of the cryogels reveals an interconnected macroporous network (4). (B) Representative confocal microscopic images of a [4% (wt/vol)] LMW HAGM cryogel of which the walls are stained with rhodamine-labelled poly-L-lysine (left) and in bright field (right). (C) Pore size of [4% (wt/vol)] LMW HAGM cryogel. 30 pores of 3 different cryogels stained with rhodamine-labelled poly-L-lysine were measured. (D) Representative scanning electron microscopy images of a 4×4×1 mm [3% (wt/vol)] BMW HAGM cryogel. Scale bar equals 1 mm (left) and 100 μm (right). (E) Injectability of alginate cryogels with or without [0.4% (wt/vol)] RGD containing 50 μg of OVA/TLR NP. (F,G) The percentage of 7AAD⁻AnnexinV⁻viable human pan T cells (F) or mouse BMDCs (G) after 24 (F) or 48 hours (F,G) culturing in medium, 3D collagen gels or cryogels. n=2-3 in 2-3 independent experiments. Values represent mean±SEM.

FIG. 2 relates to Strategy to functionalize HAGM cryogels with T cell-stimulating cues and activation of primary human T cells. (A) Approach to covalently incorporate biomolecules (pMHC complexes, antibodies or heparin) into pre-formed HAGM cryogels. (B) Representative confocal microscopic images of [4% (wt/vol)] LMW HAGM cryogels labelled with high amounts of human αCD3-A488 and human αCD28-A647. Scale bar equals 100 μm. (C,D) Fluorescence quantification of HAGM cryogels labelled with human αCD3-A488 (C) and human αCD28-A647 (D) antibodies. n=2-4 in 2-4 independent experiments. (E-G) Primary human pan T cells were stimulated with cryogels labelled with varying densities of αCD3-A488 and αCD28-A647, and the percentage of proliferated T cells (E), mean proliferation cycle (F) after 72 hours and IFNγ production (G) after 24 hours were evaluated. As positive controls, cells were stimulated with immobilized antibodies; unmodified αCD3 and αCD28 (Ab) and DBCO-fluorophore labelled αCD3 and αCD28 (DBCO Ab). n=2 in 2 independent experiments. (C-G) Values represent mean+SEM. Data were analyzed for statistical significance with a Kruskal Wallis test and Dunn's multiple comparisons test. Stars indicate significance compared to empty cryogels.

FIG. 3 relates to Functionalization of HAGM cryogels with pMHC and mouse αCD28 to stimulate mouse primary T cells. (A) Representative confocal microscopic images of [4% (wt/vol)] LMW HAGM cryogels functionalized with high amounts of mouse pMHC-A488 (H-2K^{b} SIINFEKL) and mouse αCD28-A647. Scale bar equals 100 μm. (B, C) Fluorescence quantification of HAGM cryogels labelled with mouse pMHC (B) and mouse αCD28-A647 (C) antibodies. n=3 for pMHC in 3 independent experiments and n=2 for αCD28 in 2 independent experiments. (D, E) Mouse OT-1 CD8α⁺ T cells were stimulated with cryogels labelled with varying densities of pMHC-A488 and αCD28-A647, and the mean proliferation cycle (D) after 72 hours and IFNγ production (E) after 24 hours were evaluated. As positive controls, cells were stimulated with immobilized αCD3 and αCD28 antibodies (Ab). n=2 in 2 independent experiments. (B-E) Values represent mean+SEM. Data were analyzed for statistical significance with a Kruskal Wallis test and Dunn's multiple comparisons test. Stars indicate significance compared to empty cryogels.

FIG. 4 relates to Labelling of HAGM cryogels with heparin. (A) Representative confocal microscopic images of a [4% (wt/vol)] LMW HAGM cryogel labelled with 5×10⁻⁴ equivalents of DBCO-heparin-A633 relative to carboxylic acids in the cryogel. Scale bar equals 100 μm. (B) Fluorescence quantification of HAGM cryogels labelled with 5×10⁻⁴ equivalents of DBCO-heparin-A633. n=4 in 4 independent experiments. Statistical significance with analyzed with a Kruskal Wallis test and Dunn's multiple comparisons test. Stars indicate significance compared to empty cryogels. Values represent mean+SEM.

FIG. 5 relates to Co-monomers enable biomolecule labelling of HAGM cryogels. (A) Representative confocal microscopic images of [4% (wt/vol)] LMW HAGM cryogels of 2 batches (LMW.1—unreacted GM present, LMW.2—no unreacted GM present) labelled with high amounts of human αCD3-A488. Scale bar equals 100 μm. (B) Fluorescence quantification of HAGM cryogels labelled with human αCD3-A488. n=3 for +linker, n=2 for −linker in 1 independent experiment. (C,D) Primary human pan T cells were stimulated with cryogels of batch LMW.1 (n=2 in 2 independent experiments), LMW.2 (n=3 in 3 independent experiments) or LMW.2 where HPMA was added as a co-monomer at [0.8% wt/vol)] (n=3 in 1 independent experiment). Cryogels were labelled with varying densities of αCD3-A488 and αCD28-A647, and the mean proliferation cycle (C) after 72 hours and IFNγ production (D) after 24 hours were determined. (E-F) Representative macroscopic image (E) and fluorescence quantification (F) of [4% (wt/vol)] HAGM LMW cryogels labelled with amine-Cy5 linker. n=5-10 in 2-3 independent experiments. Data was analyzed using a two-way ANOVA and Tukey's/Sidak's multiple comparisons test. Stars indicate significance compared to—, unless indicated otherwise. (G-H) Representative macroscopic image (G) and fluorescence quantification (H) of [3% (wt/vol)] HAGM HMW cryogels made with increasing amounts of GM and labelled with an amine-Cy5 linker. n=3-9 in 1-3 independent experiments. Statistical significance was tested on log-transformed data using a Kruskal Wallis test and Dunnett's multiple comparisons test. Stars indicate significance compared to [0% (wt/vol)] GM. (I) The injectability of [3% (wt/vol)] HAGM HMW cryogels through a 16 G needle was tested. Scale bar equals 4 mm. (J) Fluorescence quantification of [2.3% (wt/vol)] alginate cryogels labelled with amine-Cy5 linker. n=3 in 1 independent experiment. (B-D, F, H, J) Values represent mean±SEM. Stars indicate significance compared to empty cryogels unless indicated otherwise. (B, H, J) Data were analyzed for statistical significance with a Kruskal Wallis test and Dunn's multiple comparisons test (B,H) or one-way anova (J) on log-transformed data (H,J). (C, D, F) Statistical significance was testing using a two-way anova and Dunnett's or Sidak's multiple comparison test.

FIG. 6 is an overview of covalently attaching biomolecules to macroporous cryogels. Biocompatible polysaccharide polymers (alginate or hyaluronic acid) are chemically modified to create methacrylated polysaccharide polymers that are sensitive to free radical polymerization (1); Methacrylated polymers are dissolved in water, either with or without addition of free co-monomers such as glycidyl methacrylate (2); Free radical polymerization is triggered before freezing at −20° C. to induce ice crystal formation. The ice crystals exclude the methacrylated polymers (3); after the crosslinking of methacrylated polymers concentrated around ice crystals, thawing of the cryogels reveals an interconnected macroporous network (4); Zoom in on these networks shows that addition of co-monomers before cryogelation ensures more space between polymers within bundles of the cryogel network (5), which is pivotal for the remaining carboxylic acids (COOH) to be accessible for modification (6). When sufficient space is available to prevent steric hindrance, amino-propylamine linkers can be attached to the carboxylic acids (7) after which DBCO-functionalized biomolecules can be attached to these linkers (8), resulting in successful labelling of macroporous cryogels with a wide range of biomolecules, ranging from antibodies, protein complexes and polysaccharides (9).

FIG. 7 relates to primary human T cells can be delivered and expanded for adoptive T cell therapeutic purposes using biomolecule-functionalized HAGM cryogels. (A) Primary human pan T cells are highly viable after adhering them for 1 or 2 hours to HAGM cryogels with or without adhesion motifs (GFOGER) and/or T cell-activating biomolecules (aCD3/aCD28Ab). n=4 in 2 independent experiments. (B) Following 16 G needle-mediated injection of T cell-loaded HAGM cryogels with GFOGER+aCD3/aCD28Ab, ˜60% of 111-In-labelled T cells remain within the HAGM cryogels, and they are able to move out of the cryogel into the surrounding collagen ECM over time. n=4 in 2 independent experiments. (C) Fold expansion at day 14 of primary human CD4+ and CD8+ pan T cells with aCD3/aCD28 presented in 2D as platebound Ab or within HAGM cryogels with or without aCD3/aCD28Ab. n=4 in 2 independent experiments. (D) The multifunctionality (expression of Granzyme B, Perforin, IL-2, TNFa, IFNy) of primary human CD4+ and CD8+ pan T cells over time when expanded in 2D as platebound Ab or within HAGM cryogels with or without aCD3/aCD28Ab. n=4 in 2 independent experiments.

### DETAILED DESCRIPTION

Disclosed is a highly modular platform to functionalize 3D cryogel scaffolds by attaching biomolecules in a covalent manner. Owing to their syringe injectability, the cryogels can easily be applied in vivo.

Various biomaterial-based scaffold systems are available to present molecular cues to cells in a 3D environment, although almost all approaches do not apply covalent attachment of biomolecules. The advantage, for example, of using polysaccharide-based (e.g., hyaluronic acid or alginate) cryogels is that they are naturally non-immunogenic, biodegradable and have unique mechanical characteristics which allow for minimally invasive delivery of pre-formed constructs through injection, as opposed to many other pre-formed 3D polymer scaffolds that need to be surgically implanted. The use of pre-formed cryogels furthermore circumvents problems associated with injectable hydrogels that gel in situ including lack of control over the location of the gel, loss of cargo and a poorly defined macrostructure. So far, there are no alternative strategies reported for covalent attachment of biomolecules (e.g., antibodies, protein complexes, enzymes, DNA and polysaccharides) to these polysaccharide-based (e.g., hyaluronic acid or alginate) cryogels. As the presence of co-monomers during scaffold formation is critical to support biomolecule incorporation, this invention provides important insight to enable this approach.

Carboxylic acids are often used for bioconjugation with polymers (synthetic and natural), including hyaluronic acid and alginate. But so far this has not been performed on pre-formed 3D macroporous cryogels while preserving biofunctionality. The critical dependence on co-monomers during scaffold formation has not been reported and is unexpected.

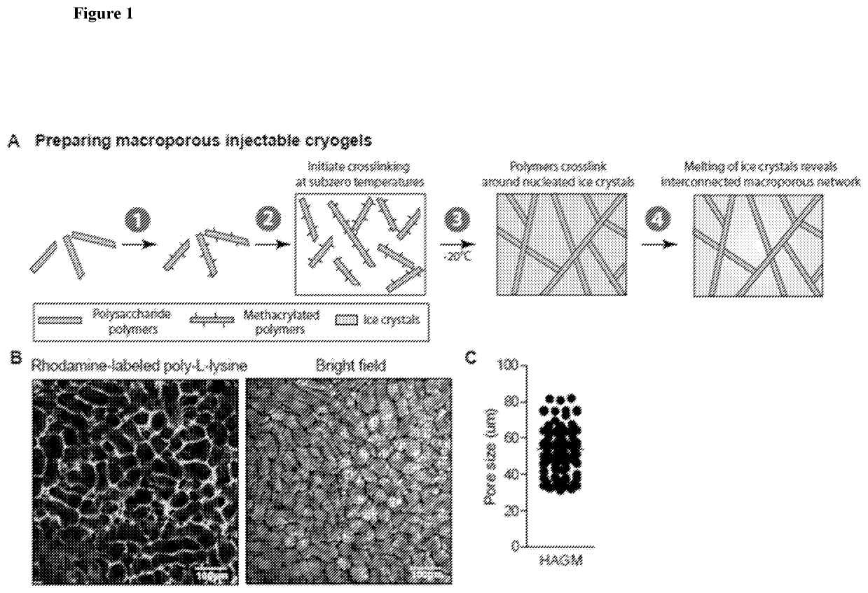

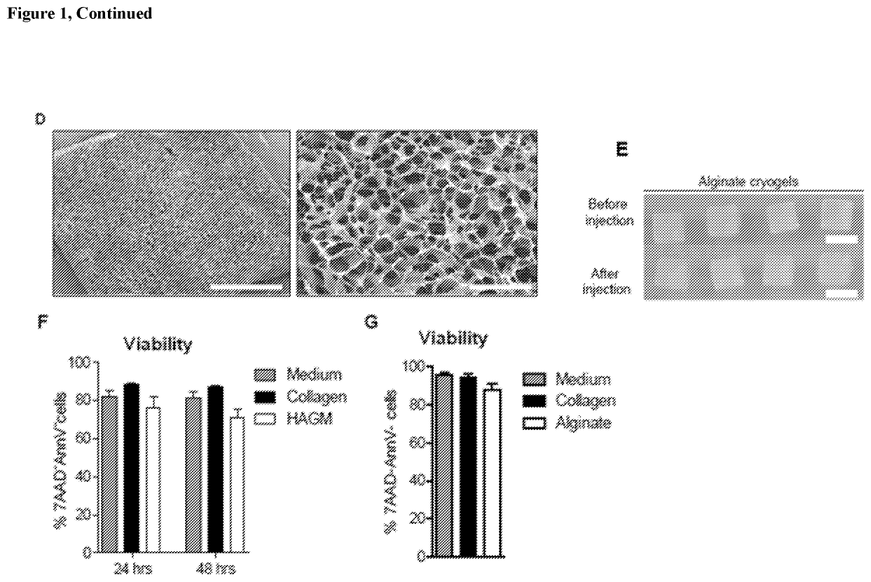

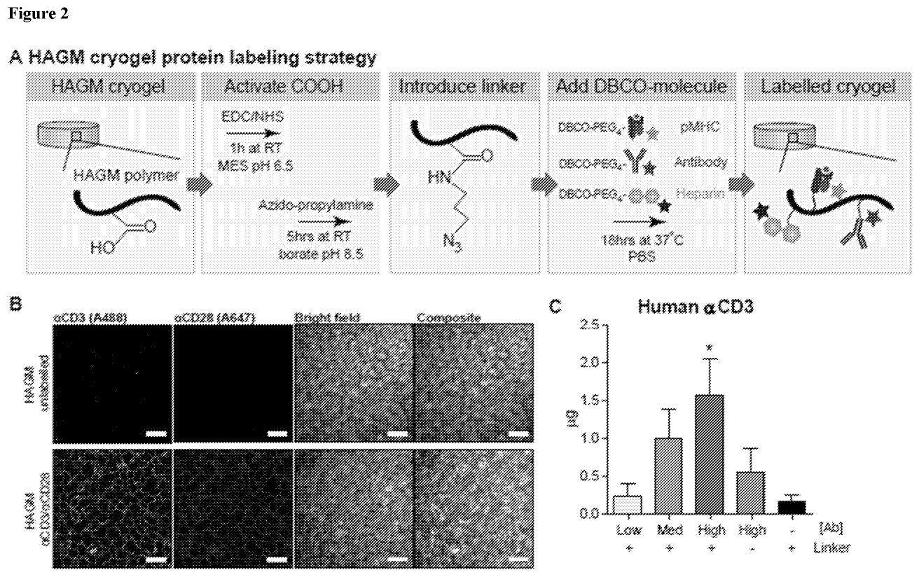

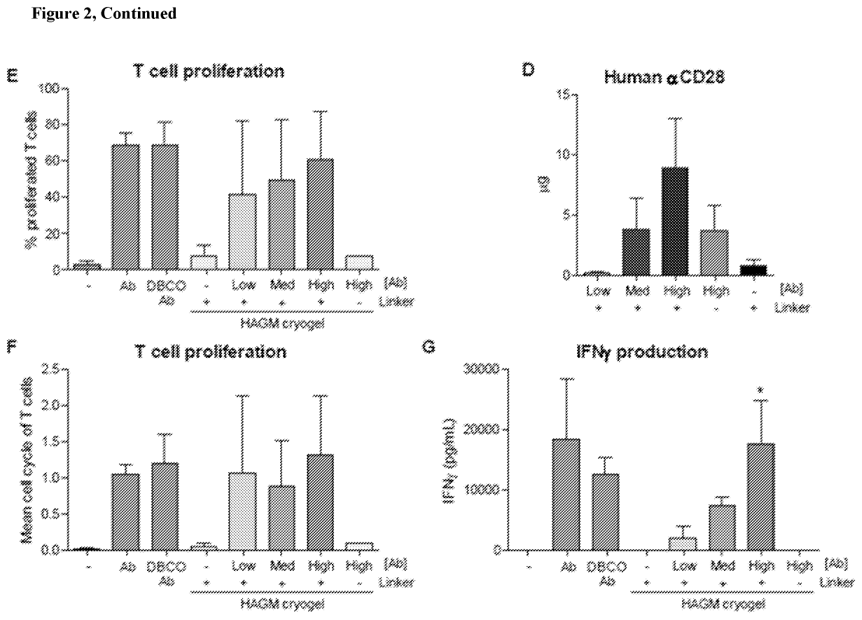

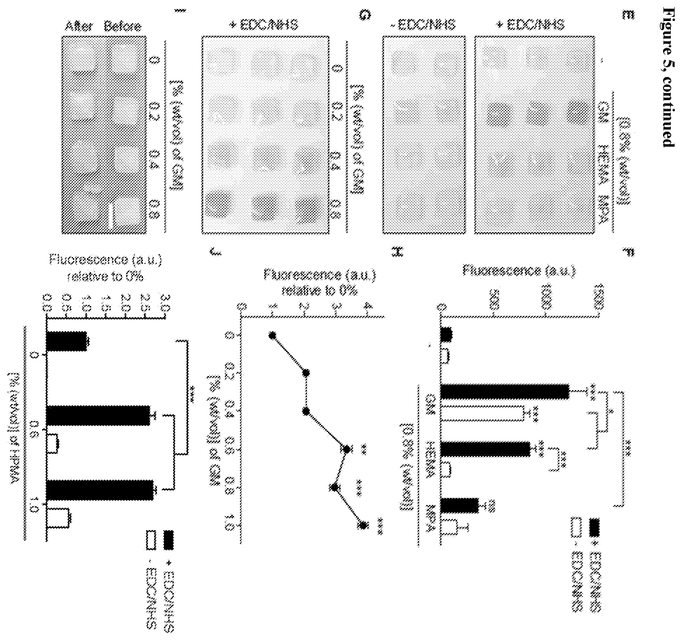

This invention focuses on covalent attachment of a wide range of biomolecules onto pre-formed polymeric cryogels. For example, macroporous cryogels based on hyaluronic acid (HA) or alginate are formed by cryogenic polymerization of methacrylated HA or alginate polymers. The resulting scaffolds are biocompatible, non-immunogenic, support cell survival and display favorable mechanical properties (FIG. 1). Here, a versatile and straightforward strategy to covalently couple activating antibodies, protein complexes and polysaccharides to these pre-formed cryogels was developed (FIG. 2-4). It has been established that the presence of co-monomers during cryogelation is required to enable and facilitate attachment of biomolecules to the cryogel post-fabrication (FIG. 5).

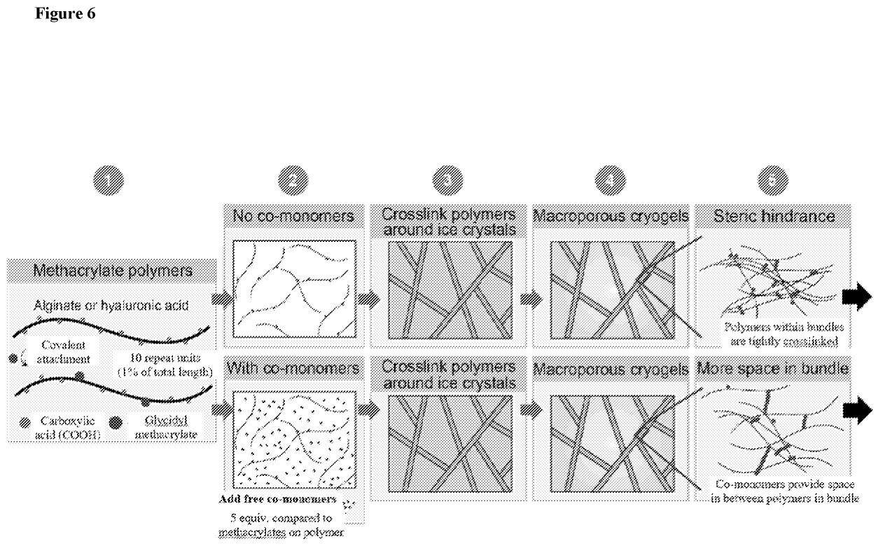

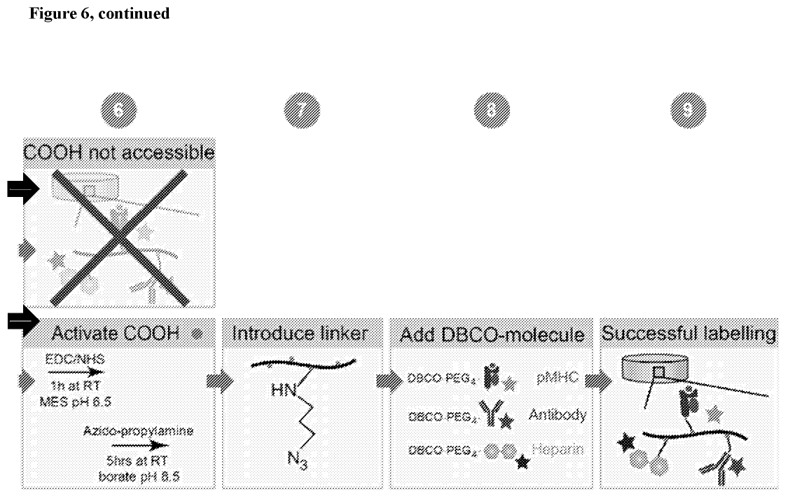

The invention is exemplified using HA/alginate cryogels, and describes a new process that enables efficient covalent attachment of biomolecules externally onto the scaffold's walls of pre-formed cryogels (FIG. 6).

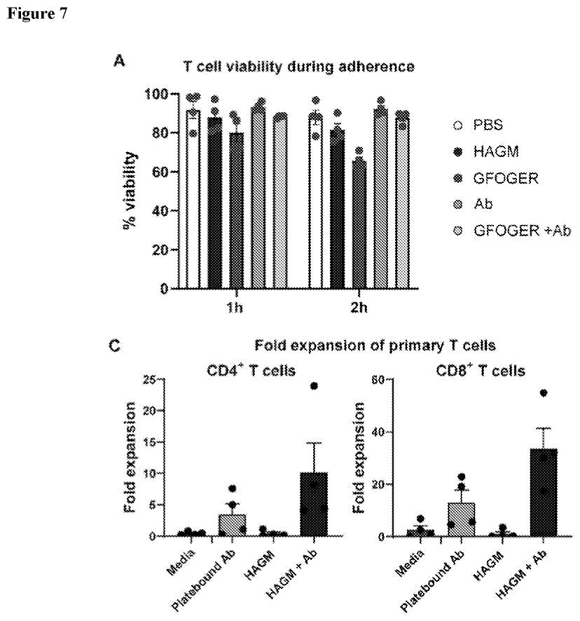

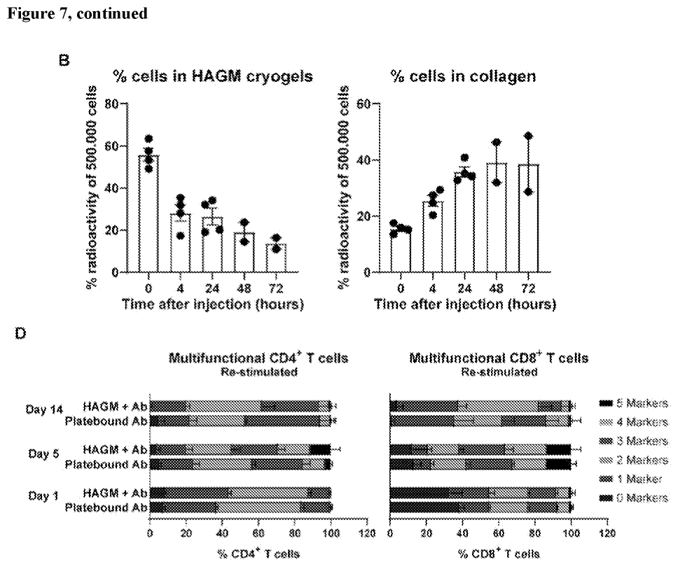

The invention can be applied, for example, for the efficient expansion of multifunctional primary T cells for adoptive T cell therapy purposes, and for the delivery of T cell-loaded activating HAGM cryogels through needle-mediated injection (FIG. 7).

U.S. Pat. No. 10,045,947 discloses injectable preformed macroscopic 3-dimensional scaffolds for minimally invasive administration (hereby incorporated by reference). U.S. Pat. No. 9,675,561 discloses injectable cryogel vaccine devices and methods of use thereof (hereby incorporated by reference).

### Definitions

The term “residue” as used herein means a portion of a chemical structure that may be truncated or bonded to another chemical moiety through any of its substitutable atoms. As an example, the structure of glycidyl methacrylate is depicted below:

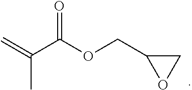

Residues of glycidyl methacrylate include, but are not limited to, any of the following structures:

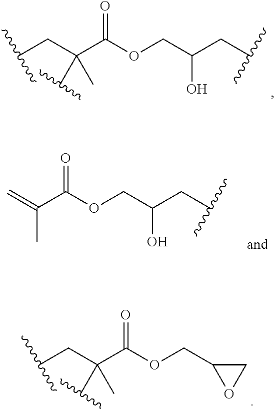 [SMILES: *CC(O)COC(=O)C(*)(C)C* ; *CC(O)COC(=O)C(=C)C ; *CC(*)(C)C(=O)OCC1CO1]

An “alkyl” group is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

The term “hydrophilic polymer” is used to mean repeating units of biological or chemical moieties that is compatible with a biological system or that mimics naturally occurring polymers. Bio-compatible polymers may be synthetic or naturally derived. Representative hydrophilic polymers include, but are not limited to oligonucleotides, polynucleotides, peptides, polypeptides, proteins, hormones, oligosaccharides, polysaccharides, lipids, glycolipids, lipopolysaccharides, phospholipids, synthetic analogues of the foregoing and combinations of the foregoing. More specifically, suitable polymers and monomers include naturally derived polymers (alginate, hyaluronic acid, chitosan, heparin, cellulose ethers (e.g. carboxymethyl cellulose, cellulose), elastin, gelatin, starch, carob gum, pectin, guar gum, carrageenan collagen, xanthan gum, fibronectin, elastin, albumin, etc.) and synthetic polymers (poly(ethylene glycol) (PEG), PEG-derivatives such as PEG-co-poly(glycolic acid; PGA) and PEG-co-poly(L-lactide; PLA), poly(2-hydroxyethyl methacrylate) (pHEMA), poly-2-hydroxyethylacrylate (polyHEA), PAAm, poly(N-isopropylacrylamide) (PNIPAAm), polyamines and polyethyleneimines, polyvinyl alcohol, polyacrylamides, polyacrylic acid, polymethacrylic acid, and so forth. Exemplary bio-compatible polymers useful in the invention include gelatin, gelatin-based bio-compatible polymers, hyaluronic acid, and hyaluronic acid-based bio-compatible polymers.

The term “crosslinking” or “crosslinked” refers to one or more chemical linkages between a compound and a polymer, two polymers (e.g., two polypeptides), or two different regions of the same polymer (e.g., two regions of one protein).

A “cryogel”, as used herein, refers to a hydrogel that has undergone cross-linking at a temperature below the solvent freezing point (e.g., 0° C. for water). As used herein, the term “hydrogel” refers to a network of polymer chains (e.g., recombinant proteins) in which water or a solvent acts as a dispersion medium. In some embodiments, hydrogels have tunable mechanical properties which are not possible to achieve with other compositions, such as biofilms. In some embodiments, a hydrogel may be self-healing, in that the hydrogel can be broken apart and put back together. In other words, dried pieces of a hydrogel can be rehydrated and assembled together using the re-hydrated gel as a “glue.”

When used in a polymeric linking moiety, polyethylene glycol can consist of 2 repeat units of ethylene glycol up to 500,000 repeat units of ethylene glycol. The average molecular weight of the PEG moiety may be about 100 Da to about 10,000 Da, about 500 Da to about 5000 Da, about 1000 Da to about 5000 Da, about 2000 Da to about 5000 Da, or about 3500 Da.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

The formulations comprising a cryogel of the invention, which formulations are described hereinbelow, may optionally contain a pharmaceutically acceptable excipient.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

The present invention also contemplates pharmaceutically acceptable salts of the compounds of the invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

### Embodiments of the Invention

In certain embodiments, the present invention provides a polymer comprising a moiety of formula (I):

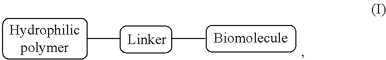

wherein the hydrophilic polymer is crosslinked to one or more additional hydrophilic polymer molecules, and the linker is covalently attached to the hydrophilic polymer.

In certain embodiments, the hydrophilic polymer is a synthetic polymer or a polysaccharide, protein or peptide. In certain embodiments, the hydrophilic polymer is a polysaccharide. In certain embodiments, the polysaccharide is selected from hyaluronic acid, alginic acid, chitosan, dextran, heparin and hydroxyethylcellulose. In certain embodiments, the polysaccharide is a polyuronic acid. In certain embodiments, the polysaccharide is hyaluronic acid or alginic acid.

In certain embodiments, the crosslinks are covalent. In certain embodiments, the polymer is crosslinked via acrylate or methacrylate residues. In certain embodiments, the crosslinks are derived from glycidyl methacrylate residues.

In certain embodiments, the linker is covalently attached to the hydrophilic polymer via a carboxyl group. In certain embodiments, the linker comprises one or more groups selected from alkyl, amide, triazole and polyether. In certain embodiments, the linker comprises a residue derived from dibenzocyclooctyne (DBCO). In certain embodiments, the linker comprises a hydrophilic polymer. In certain embodiments, the linker comprises a polyethylene glycol (PEG) group. In certain embodiments, the polyethylene glycol (PEG) group has a molecular weight of from about 0.5 to about 50 kDa. In certain embodiments, the polyethylene glycol (PEG) group has a molecular weight of about 3 kDa. In certain embodiments, the linker comprises a residue derived from azido-propylamine. In certain embodiments, the linker comprises a residue derived from dibenzocyclooctyne-PEG4-N-hydroxysuccinimidyl ester or dib enzocyclooctyne-PEG4-amine.

In certain embodiments, the biomolecule is selected from antibodies, protein complexes enzymes, DNA and polysaccharides. In certain embodiments, the biomolecule is capable of promoting cell expansion. In certain embodiments, the cells are non-immune cells. In certain embodiments, wherein the cells are stem cells. In certain embodiments, the cells are immune cells. In certain embodiments, the cells are selected from T cells, NK cells and dendritic cells. In certain embodiments, the cells are T cells. In certain embodiments, the biomolecule is selected from heparin, a CD3 antibody, a CD28 antibody and a peptide-major histocompatibility complex (pMHC).

In certain embodiments, the invention provides a cryogel comprising a polymer of any one of the preceding claims. In certain embodiments, the invention provides a method of expanding cells, comprising contacting one or more cells with a polymer or a cryogel of the invention. In certain embodiments, the cells are T cells.

In certain embodiments, the present invention provides a method of making a cryogel, comprising crosslinking a hydrophilic polymer in an aqueous solvent to generate a crosslinked polymer. In certain embodiments, the hydrophilic polymer is a polysaccharide. In certain embodiments, the polysaccharide is acrylated or methacrylated.

In certain embodiments, the acrylated or methacrylated polysaccharide is reacted with an acrylate or methacrylate co-monomer. In certain embodiments, the molar ratio of acrylate or methacrylate co-monomer to acrylate or methacrylate groups in the acrylated or methacrylated polysaccharide is at least about 0.1:1. In certain embodiments, the molar ratio of acrylate or methacrylate co-monomer to acrylate or methacrylate groups in the acrylated or methacrylated polysaccharide is from about 0.1:1 to about 30:1. In certain embodiments, the molar ratio of acrylate or methacrylate co-monomer to acrylate or methacrylate groups in the acrylated or methacrylated polysaccharide is about 1:1 to 20:1. In certain embodiments, the acrylate or methacrylate co-monomer is glycidyl methacrylate. In certain embodiments, the polysaccharide is selected from hyaluronic acid, alginic acid, chitosan, dextran, heparin and hydroxyethylcellulose. In certain embodiments, the acrylated or methacrylated polysaccharide is hyaluronic acid methacrylate (HAGM) or alginate methacrylate. In certain embodiments, the degree of methacrylation of the polysaccharide from about 1 to 90 mol %.

In certain embodiments, the acrylated or methacrylated polysaccharide is reacted with the acrylate or methacrylate co-monomer in the presence of a radical initiator. In certain embodiments, the aqueous solvent is frozen after the acrylated or methacrylated polysaccharide is contacted with the radical initiator (such as a redox initiator (e.g., ammonium persulfate/tetramethylethylenediamine (APS/TEMED)) or a photoinitiator (e.g., Irgacure 2959).

In certain embodiments, the cryogel is crosslinked by polycondensation, click-chemistry, Michael-type addition or enzymatically. In certain embodiments, the cryogel is crosslinked by click-chemistry. In certain embodiments, the cryogel is physically and/or non-covalently crosslinked by e.g., peptide-peptide, ionic and/or hydrophobic interactions.

In certain embodiments, the crosslinked polymer is reacted with a linker that comprises an azide, alkyne, alkene or thiol group. In certain embodiments, the crosslinked polymer is reacted with azido-terminated molecule such as azido-amine derivatives (azido-PEG-amine, azido, ethylamine, etc) or azido-alcohol derivatives (azido-PEG-amine, azido-propanol, etc) or with moieties that contain alkene, alkyne or thiol groups. In certain embodiments, the crosslinked polymer is reacted with an azido-propylamine in the presence of a coupling system. In certain embodiments, the coupling system comprises one or more aminium, phosphonium, carbodiimide or N-hydroxy reagents. In certain embodiments, the coupling system comprises N-hydroxysuccinimide and ethyl(dimethylaminopropyl) carbodiimide.

In certain embodiments, the crosslinked polymer is reacted with a biomolecule that is conjugated to a dibenzocyclooctyne (DBCO) moiety. In certain embodiments, the invention provides cryogel prepared according to the method of the invention.

In certain embodiments, the invention provides a method of expanding cells, comprising contacting one or more cells with a cryogel of the invention. In certain embodiments, the cells are non-immune cells. In certain embodiments, the cells are stem cells. In certain embodiments, the cells are immune cells. In certain embodiments, the cells are selected from T cells, NK cells and dendritic cells. In certain embodiments, the cells are T cells.

In certain embodiments, the invention provides a cryogel of the invention and a pharmaceutically acceptable carrier. In certain embodiments, the formulation is injectable.

In certain embodiments, the invention provides a method of delivering a biomolecule to a tissue, comprising contacting the tissue with the formulation of the invention. In certain embodiments, the invention provides a method of delivering activated T-cells to a tissue, comprising contacting the tissue with a formulation or cryogel of the invention.

The shape of the cryogel is dictated by a mold and can thus take on any shape desired by the fabricator, e.g., various sizes and shapes (disc, cylinders, squares, cubes, spheres, fibers, strings, foam, etc.) are prepared by cryogenic polymerization. Injectable cryogels can be prepared in the micrometer-scale to centimeter-scale. For instance, cube-shaped (i.e., cubiform) cryogels (4×4×1, 5×5×1, or 10×10×1 mm³) were fabricated and injected through a standard 16 G hypodermic needle.

The invention allows for covalent attachment of biomolecules that are presented externally on polymer's walls of 3D macroporous biomaterial-based scaffolds, instead of non-covalent methods of presenting biomolecules on these scaffolds (via ionic interactions, hydrophobic interactions, physical entrapment, etc.). The labelling method that is proposed is highly modular, efficient and is dependent on the presence of co-monomers during cryogel fabrication (FIG. 5).

The cryogels of the invention may be useful as 3D culture systems to provide cells with stimulatory/survival cues; as tools to study ex vivo interaction of cells and molecular cues in a controlled context. Enhance immunotherapeutic approaches: e.g. ex vivo (T) cell expansion, in vivo (immune) cell stimulation.

### Exemplary Advantages of the Invention

The advantages of the materials disclosed herein include: high modularity; efficient and easy to work with; easy to wash away potential toxic molecules used for labelling; biomolecules attached in a covalent manner and presented externally on the scaffold's walls; bioavailability of molecules is retained as molecules are not exposed to freeze/thawing and free-radical polymerization during cryogel formation (which happens when molecules are physically entrapped); versatile platform for production of cryogels. Any water soluble polymers (synthetic and natural) and monomers can potentially be used.

### EXAMPLES

### Example 1

FIG. 1 relates to Pre-formed cryogels are macroporous, injectable and support cell survival. (A) Schematic overview of the cryogelation process to produce injectable cryogels. Polysaccharide polymers (alginate or hyaluronic acid) are chemically modified to create methacrylated polysaccharide polymers that are sensitive to free radical polymerization (1); Methacrylated polymers are dissolved in water (2); Free radical polymerization is triggered before freezing at −20° C. to induce ice crystal formation. The ice crystals exclude the methacrylated polymers (3); after the crosslinking of methacrylated polymers concentrated around ice crystals, thawing of the cryogels reveals an interconnected macroporous network (4). (B) Representative confocal microscopic images of a [4% (wt/vol)] LMW HAGM cryogel of which the walls are stained with rhodamine-labelled poly-L-lysine (left) and in bright field (right). (C) Pore size of [4% (wt/vol)] LMW

HAGM cryogel. 30 pores of 3 different cryogels stained with rhodamine-labelled poly-L-lysine were measured. (D) Representative scanning electron microscopy images of a 4×4×1 mm [3% (wt/vol)] BMW HAGM cryogel. Scale bar equals 1 mm (left) and 100 μm (right). (E) Injectability of alginate cryogels with or without [0.4% (wt/vol)] RGD containing 50 μg of OVA/TLR NP. (F,G) The percentage of 7AAD⁻AnnexinV⁻viable human pan T cells

(F) or mouse BMDCs (G) after 24 (F) or 48 hours (F,G) culturing in medium, 3D collagen gels or cryogels. n=2-3 in 2-3 independent experiments. Values represent mean±SEM.

### Example 2

FIG. 2 relates to Strategy to functionalize HAGM cryogels with T cell-stimulating cues and activation of primary human T cells. (A) Approach to covalently incorporate biomolecules (pMHC complexes, antibodies or heparin) into pre-formed HAGM cryogels. (B) Representative confocal microscopic images of [4% (wt/vol)] LMW HAGM cryogels labelled with high amounts of human αCD3-A488 and human αCD28-A647. Scale bar equals 100 μm. (C,D) Fluorescence quantification of HAGM cryogels labelled with human αCD3-A488 (C) and human αCD28-A647 (D) antibodies. n=2-4 in 2-4 independent experiments. (E-G) Primary human pan T cells were stimulated with cryogels labelled with varying densities of αCD3-A488 and αCD28-A647, and the percentage of proliferated T cells (E), mean proliferation cycle (F) after 72 hours and IFNγ production (G) after 24 hours were evaluated. As positive controls, cells were stimulated with immobilized antibodies; unmodified αCD3 and αCD28 (Ab) and DBCO-fluorophore labelled αCD3 and αCD28 (DBCO Ab). n=2 in 2 independent experiments. (C-G) Values represent mean+SEM. Data were analyzed for statistical significance with a Kruskal Wallis test and Dunn's multiple comparisons test. Stars indicate significance compared to empty cryogels.

### Example 3

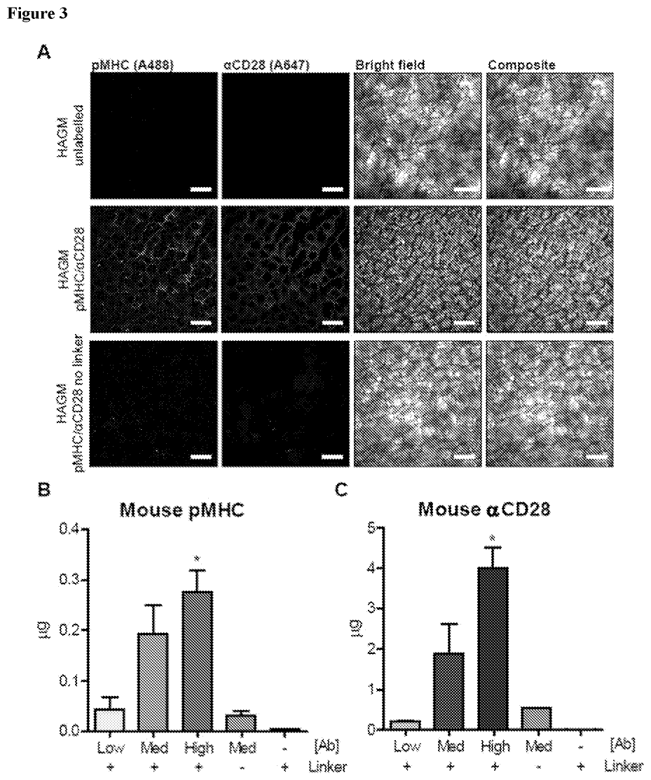

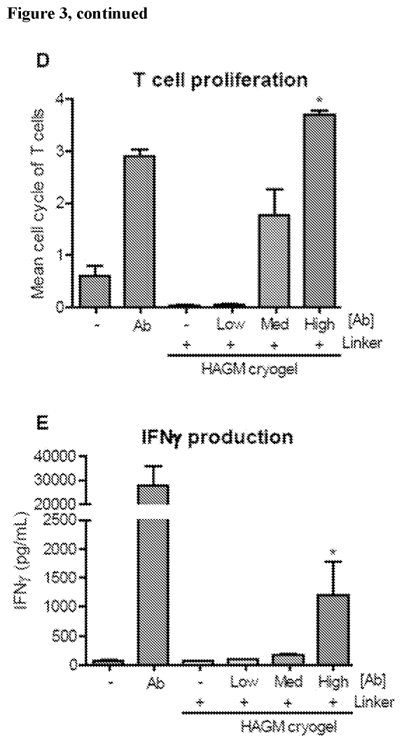

FIG. 3 relates to Functionalization of HAGM cryogels with pMHC and mouse αCD28 to stimulate mouse primary T cells. (A) Representative confocal microscopic images of [4% (wt/vol)] LMW HAGM cryogels functionalized with high amounts of mouse pMHC-A488 (H-2K^{b} SIINFEKL) and mouse αCD28-A647. Scale bar equals 100 μm. (B, C) Fluorescence quantification of HAGM cryogels labelled with mouse pMHC (B) and mouse αCD28-A647 (C) antibodies. n=3 for pMHC in 3 independent experiments and n=2 for αCD28 in 2 independent experiments. (D, E) Mouse OT-1 CD8α⁺ T cells were stimulated with cryogels labelled with varying densities of pMHC-A488 and αCD28-A647, and the mean proliferation cycle (D) after 72 hours and IFNγ production (E) after 24 hours were evaluated. As positive controls, cells were stimulated with immobilized αCD3 and αCD28 antibodies (Ab). n=2 in 2 independent experiments. (B-E) Values represent mean+SEM. Data were analyzed for statistical significance with a Kruskal Wallis test and Dunn's multiple comparisons test. Stars indicate significance compared to empty cryogels.

### Example 4

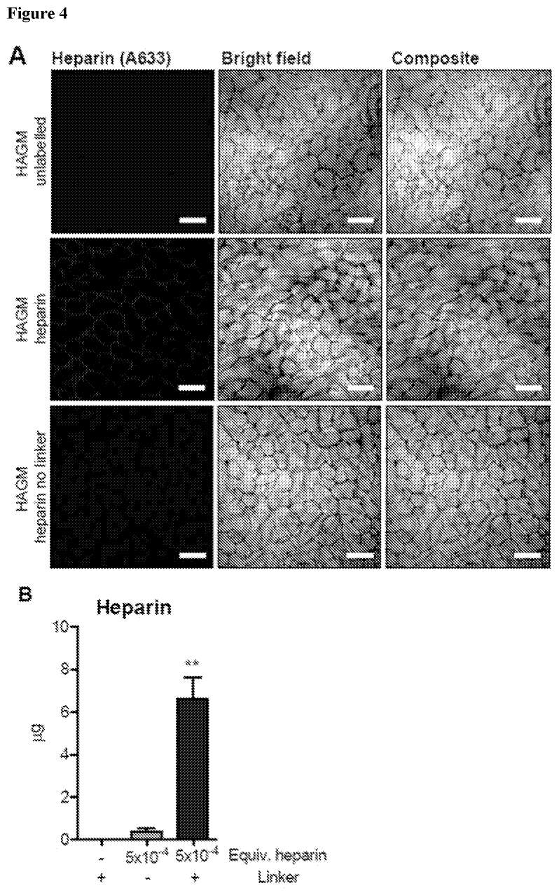

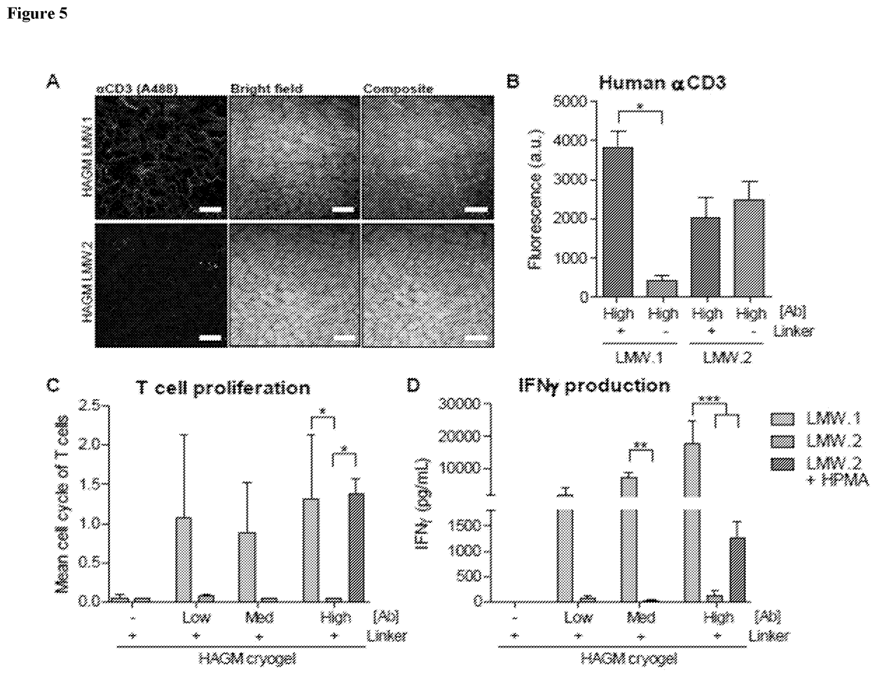

FIG. 4 relates to Labelling of HAGM cryogels with heparin. (A) Representative confocal microscopic images of a [4% (wt/vol)] LMW HAGM cryogel labelled with 5×10⁻⁴ equivalents of DBCO-heparin-A633 relative to carboxylic acids in the cryogel. Scale bar equals 100 μm. (B) Fluorescence quantification of HAGM cryogels labelled with 5×10⁻⁴ equivalents of DBCO-heparin-A633. n=4 in 4 independent experiments. Statistical significance with analyzed with a Kruskal Wallis test and Dunn's multiple comparisons test. Stars indicate significance compared to empty cryogels. Values represent mean+SEM.

### Example 5

FIG. 5 relates to Co-monomers enable biomolecule labelling of HAGM cryogels. (A) Representative confocal microscopic images of [4% (wt/vol)] LMW HAGM cryogels of 2 batches (LMW.1—unreacted GM present, LMW.2—no unreacted GM present) labelled with high amounts of human αCD3-A488. Scale bar equals 100 μm. (B)

Fluorescence quantification of HAGM cryogels labelled with human αCD3-A488. n=3 for +linker, n=2 for −linker in 1 independent experiment. (C,D) Primary human pan T cells were stimulated with cryogels of batch LMW.1 (n=2 in 2 independent experiments), LMW.2 (n=3 in 3 independent experiments) or LMW.2 where HPMA was added as a co-monomer at [0.8% wt/vol)] (n=3 in 1 independent experiment). Cryogels were labelled with varying densities of αCD3-A488 and αCD28-A647, and the mean proliferation cycle (C) after 72 hours and IFNγ production (D) after 24 hours were determined. (E-F) Representative macroscopic image (E) and fluorescence quantification (F) of [4% (wt/vol)] HAGM LMW cryogels labelled with amine-Cy5 linker. n=5-10 in 2-3 independent experiments. Data was analyzed using a two-way ANOVA and Tukey's/Sidak's multiple comparisons test. Stars indicate significance compared to—, unless indicated otherwise. (G-H) Representative macroscopic image (G) and fluorescence quantification (H) of [3% (wt/vol)] HAGM HMW cryogels made with increasing amounts of GM and labelled with an amine-Cy5 linker. n=3-9 in 1-3 independent experiments. Statistical significance was tested on log-transformed data using a Kruskal Wallis test and Dunnett's multiple comparisons test. Stars indicate significance compared to [0% (wt/vol)] GM. (I) The injectability of [3% (wt/vol)] HAGM HMW cryogels through a 16 G needle was tested. Scale bar equals 4 mm. (J) Fluorescence quantification of [2.3% (wt/vol)] alginate cryogels labelled with amine-Cy5 linker. n=3 in 1 independent experiment.

(B-D, F, H, J) Values represent mean±SEM. Stars indicate significance compared to empty cryogels unless indicated otherwise. (B, H, J) Data were analyzed for statistical significance with a Kruskal Wallis test and Dunn's multiple comparisons test (B,H) or one-way anova (J) on log-transformed data (H,J). (C, D, F) Statistical significance was testing using a two-way anova and Dunnett's or Sidak's multiple comparison test.

### Example 6

FIG. 6 relates to Overview of invention to covalently attach biomolecules to macroporous cryogels. Biocompatible polysaccharide polymers (alginate or hyaluronic acid) are chemically modified to create methacrylated polysaccharide polymers that are sensitive to free radical polymerization (1); Methacrylated polymers are dissolved in water, either with or without addition of free co-monomers such as glycidyl methacrylate (2); Free radical polymerization is triggered before freezing at −20° C. to induce ice crystal formation. The ice crystals exclude the methacrylated polymers (3); after the crosslinking of methacrylated polymers concentrated around ice crystals, thawing of the cryogels reveals an interconnected macroporous network (4); Zoom in on these networks shows that addition of co-monomers before cryogelation ensures more space between polymers within bundles of the cryogel network (5), which is pivotal for the remaining carboxylic acids (COOH) to be accessible for modification (6). When sufficient space is available to prevent steric hindrance, amino-propylamine linkers can be attached to the carboxylic acids (7) after which DBCO-functionalized biomolecules can be attached to these linkers (8), resulting in successful labelling of macroporous cryogels with a wide range of biomolecules, ranging from antibodies, protein complexes and polysaccharides (9).

### Example 7

FIG. 7 relates to Primary human T cells can be delivered and expanded for adoptive T cell therapeutic purposes using biomolecule-functionalized HAGM cryogels. (A) Primary human pan T cells are highly viable after adhering them for 1 or 2 hours to HAGM cryogels with or without adhesion motifs (GFOGER) and/or T cell-activating biomolecules (aCD3/aCD28Ab). n=4 in 2 independent experiments. (B) Following 16 G needle-mediated injection of T cell-loaded HAGM cryogels with GFOGER+aCD3/aCD28Ab, ˜60% of 111-In-labelled T cells remain within the HAGM cryogels, and they are able to move out of the cryogel into the surrounding collagen ECM over time. n=4 in 2 independent experiments. (C) Fold expansion at day 14 of primary human CD4+ and CD8+ pan T cells with aCD3/aCD28 presented in 2D as platebound Ab or within HAGM cryogels with or without aCD3/aCD28Ab. n=4 in 2 independent experiments. (D) The multifunctionality (expression of Granzyme B, Perforin, IL-2, TNFa, IFNy) of primary human CD4+ and CD8+ pan T cells over time when expanded in 2D as platebound Ab or within HAGM cryogels with or without aCD3/aCD28Ab. n=4 in 2 independent experiments.

### INCORPORATION BY REFERENCE

All of the U.S. patents and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A polymer comprising a moiety of formula (I): 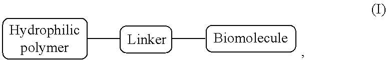
wherein the hydrophilic polymer is crosslinked to one or more additional hydrophilic polymer molecules, and the linker is covalently attached to the hydrophilic polymer.

2. The polymer of claim 1, wherein the hydrophilic polymer is a synthetic polymer or a polysaccharide, protein or peptide.

3. The polymer of claim 2, wherein the hydrophilic polymer is a polysaccharide.

4. The polymer of claim 3, wherein the polysaccharide is selected from hyaluronic acid, alginic acid, chitosan, dextran, heparin and hydroxyethylcellulose.

5. The polymer of claim 4, wherein the polysaccharide is a polyuronic acid.

6. The polymer of claim 4, wherein the polysaccharide is hyaluronic acid or alginic acid.

7. The polymer of any one of the preceding claims, wherein the crosslinks are covalent.

8. The polymer of any one of the preceding claims, wherein the polymer is crosslinked via acrylate or methacrylate residues.

9. The polymer of any one of the preceding claims, wherein the crosslinks are derived from glycidyl methacrylate residues.

10. The polymer of any one of the preceding claims, wherein the linker is covalently attached to the hydrophilic polymer via a carboxyl group.

11. The polymer of any one of the preceding claims, wherein the linker comprises one or more groups selected from alkyl, amide, triazole and polyether.

12. The polymer of any one of the preceding claims, wherein the linker comprises a residue derived from dibenzocyclooctyne (DBCO).

13. The polymer of any one of the preceding claims, wherein the linker comprises a hydrophilic polymer.

14. The polymer of claim 13, wherein the linker comprises a polyethylene glycol (PEG) group.

15. The polymer of claim 14, wherein the polyethylene glycol (PEG) group has a molecular weight of from about 0.5 to about 50 kDa.

16. The polymer of claim 15, wherein the polyethylene glycol (PEG) group has a molecular weight of about 3 kDa.

17. The polymer of any one of claims 1-16, wherein the linker comprises a residue derived from azido-propylamine.

18. The polymer of any one of claims 1-17, wherein the linker comprises a residue derived from dibenzocyclooctyne-PEG4-N-hydroxysuccinimidyl ester or dibenzocyclooctyne-PEG4-amine.

19. The polymer of any one of claims 1-18, wherein the biomolecule is selected from antibodies, protein complexes enzymes, DNA and polysaccharides.

20. The polymer of any one of claims 1-19, wherein the biomolecule is capable of promoting cell expansion.

21. The polymer of claim 20, wherein the cells are non-immune cells.

22. The polymer of claim 21, wherein the cells are stem cells.

23. The polymer of claim 20, wherein the cells are immune cells.

24. The polymer of claim 23, wherein the cells are selected from T cells, NK cells and dendritic cells.

25. The polymer of claim 24, wherein the cells are T cells.

26. The polymer of any one of claims 1-25, wherein the biomolecule is selected from heparin, a CD3 antibody, a CD28 antibody and a peptide-major histocompatibility complex (pMHC).

27. A cryogel comprising a polymer of any one of claims 1-26.

28. A method of expanding cells, comprising contacting one or more cells with a polymer of claims 26 or a cryogel of claim 27.

29. The method of claim 28, wherein the cells are T cells.

30. A method of making a cryogel, comprising crosslinking a hydrophilic polymer in an aqueous solvent to generate a crosslinked polymer.

31. The method of claim 30, wherein the hydrophilic polymer is a polysaccharide.

32. The method of claim 31, wherein the polysaccharide is acrylated or methacrylated.

33. The method of claim 32, wherein the acrylated or methacrylated polysaccharide is reacted with an acrylate or methacrylate co-monomer.

34. The method of claim 33, wherein the molar ratio of acrylate or methacrylate co-monomer to acrylate or methacrylate groups in the acrylated or methacrylated polysaccharide is at least about 0.1:1.

35. The method of claim 33 or 34, wherein the molar ratio of acrylate or methacrylate co-monomer to acrylate or methacrylate groups in the acrylated or methacrylated polysaccharide is from about 0.1:1 to about 30:1.

36. The method of any one of claims 33-35, wherein the molar ratio of acrylate or methacrylate co-monomer to acrylate or methacrylate groups in the acrylated or methacrylated polysaccharide is about 1:1 to 20:1.

37. The method of any one of claims 33-36, wherein the acrylate or methacrylate co-monomer is glycidyl methacrylate.

38. The method of any one of claims 31-37, wherein the polysaccharide is selected from hyaluronic acid, alginic acid, chitosan, dextran, heparin and hydroxyethylcellulose

39. The method of any one of claims 32-38, wherein the acrylated or methacrylated polysaccharide is hyaluronic acid methacrylate (HAGM) or alginate methacrylate.

40. The method of claim 33, wherein the acrylated or methacrylated polysaccharide is reacted with the acrylate or methacrylate co-monomer in the presence of a radical initiator.

41. The method of claim 40, wherein the aqueous solvent is frozen after the acrylated or methacrylated polysaccharide is contacted with the radical initiator.

42. The method of any one of claims 30-41, wherein the crosslinked polymer is reacted with a linker that comprises an azide, alkyne, alkene or thiol group.

43. The method of any one of claims 30-42, wherein the crosslinked polymer is reacted with an azido-propylamine in the presence of a coupling system.

44. The method of any one of claims 30-43, wherein the crosslinked polymer is reacted with a biomolecule that is conjugated to an azide, alkyne, alkene or thiol group.

45. The method of any one of claims 30-44, wherein the crosslinked polymer is reacted with a biomolecule that is conjugated to a dibenzocyclooctyne (DBCO) moiety.

46. A cryogel prepared according to the method of any one of claims 30-45.

47. A method of expanding cells, comprising contacting one or more cells with a cryogel of claim 46.

48. The method of claim 47, wherein the cells are non-immune cells.

49. The method of claim 48, wherein the cells are stem cells.

50. The method of claim 49, wherein the cells are immune cells.

51. The method of claim 50, wherein the cells are selected from T cells, NK cells and dendritic cells.

52. The method of claim 51, wherein the cells are T cells.

53. A formulation, comprising the cryogel of claim 27 or 46; and a pharmaceutically acceptable carrier.

54. The formulation of claim 53, wherein the formulation is injectable.

55. A method of delivering a biomolecule to a tissue, comprising contacting the tissue with the formulation of claim 53.

56. A method of delivering activated T-cells to a tissue, comprising contacting the tissue with the formulation of claim 53 or the cryogel of claim 27 or 46.
